(19) United States
(12) Patent Application Publication
(10) Pub. No.: US 2010/0087852 A1
(43) Pub. Date: Apr. 8, 2010

(54) TEMPORARY SURGICAL CLOSURE FOR A BODY CAVITY

(76) Inventors: Michael L. Cheatham, Orlando, FL (US); Perry W. Croll, Salt Lake City, UT (US); Timothy R. Wolfe, Salt Lake City, UT (US); Marshall T. Denton, Salt Lake City, UT (US); J. Michael Brown, Salt Lake City, UT (US); Mark A. Christensen, Salt Lake City, UT (US)

Correspondence Address: TRASKBRITT, P.C., P.O. BOX 2550, SALT LAKE CITY, UT, 84110, US

(21) Appl. No.: 12/450,084

(22) PCT Filed: Mar. 14, 2008
(86) PCT No.: PCT/IB2008/002453

§ 371 Date: Sep. 10, 2009

Related U.S. Application Data
(60) Provisional application No. 60/918,385, filed on Mar. 16, 2007.

(51) Int. Cl. A61B 17/08; A61B 1/00

(52) U.S. Cl. 606/213; 600/184

(57) ABSTRACT

Described is an apparatus (100) adapted to temporarily close a surgical access opening (106) to a patient's body cavity (110). The apparatus includes a base (102) that is disposed around a perimeter of the opening, and a cap (104) that typically provides resealable access through the opening. The base generally forms an air-resistant seal to the patient's skin. In certain embodiments, the cap may be removable from the base. The base typically includes structure (180) configured to permit suction to be applied effective to remove undesired fluids from the body cavity. A cap is adapted to change in size to accommodate expansion and contraction of viscera.

### PRIORITY CLAIM

This application claims the benefit under 35 U.S.C. §119(e) of the filing date of U.S. Provisional Patent Application Ser. No. 60/918,385, filed Mar. 16, 2007, for “TEMPORARY SURGICAL CLOSURE FOR A BODY CAVITY”, the contents of the entirety of which are incorporated by this reference.

### TECHNICAL FIELD

The invention relates generally to surgical apparatus, and particularly to a temporary, resealable, post-operative closure for a medical patient's body cavity.

### BACKGROUND

Catastrophic abdominal events, such as trauma, hemorrhage, sepsis, or infection, may lead to elevated intra-abdominal pressure (IAP), which can progress to abdominal compartment syndrome (ACS). ACS constitutes a life-threatening condition. Essentially, the afflicted patient's internal organs become engorged with fluids and consequently expand to collectively occupy a larger volume than the abdominal compartment can normally accommodate. Consequently, the pressure inside the abdominal compartment rises to detrimental levels. Sustained ACS leads to organ failure and patient death. Treatment of ACS may include opening, and/or leaving open, the abdominal wall until the elevated pressure subsides.

Treatment of certain medical conditions may require a plurality of surgeries spaced apart by periods of time. Often, such surgeries would require re-opening the same surgical opening to gain entrance to a patient's body cavity, such as the thoracic or abdominal cavity. Such repetitive surgery can cause complications arising from multiple re-opening of a conventionally closed surgical incision site. For example, tissue at the edge of the incision may become damaged, or tattered from previous sutures, and therefore fail to hold a sufficient number of sutures required to maintain a subsequent closure.

Medical conditions exist in which a temporary closure would provide a beneficial treatment option for a patient. Considerable effort has been expended by persons skilled in the medical art to provide such a temporary closure. Certain relevant United States patents include: U.S. Pat. Nos. 6,951,553; 6,936,037; 6,637,453; 6,626,891; and 4,969,880. Certain relevant United States patent applications include: 2006/0241689; 2006/0189910; 2006/0079852; 2005/0283105; 2005/0234510; 2005/0177190; 2005/0034732; 2004/0221431; 2002/0017304, which disclose medically appropriate materials, structure, and methods related to devices adapted for use in a medical patient, the contents of the entirety of each of which are incorporated by this reference.

In U.S. Pat. No. 3,026,874, the contents of the entirety of which are incorporated by this reference, issued Mar. 27, 1962, Stevens discloses a wound shield adapted to form an enclosure surrounding a wound. The enclosure includes a perimeter dam covered by a shield. The dam and closure may be held in position by adhering belt extensions to the skin proximal the wound. Provision is made to permit irrigating the wound.

In United States Patent Application 2005/0085757, published Apr. 21, 2005, Santanello, the contents of the entirety of which are incorporated by this reference, discloses an expandable temporary abdominal closure. Santanello's closure includes a pre-folded sheet of material that may be placed over a surgical opening to shield the site from contamination. The closure may be bonded or sutured to the skin or fascia around the opening. Certain embodiments may also permit visual inspection while relieving abdominal pressure. Folds in the sheet of material are arranged to permit an accordion-like expansion to provide a shielded extension that effectively enlarges the abdominal cavity and relieves abdominal pressure. A resealable seam may be included in certain embodiments to permit access to the abdominal compartment without removing the closure from the patient. Bilateral wings, which may be tucked under the thoracic wall to resist adhesion between the viscera and thoracic wall, may be included in certain embodiments.

In United States Patent Application 2004/0030304, published Feb. 12, 2004, Hunt et al., the contents of the entirety of which are incorporated by this reference, disclose an abdominal wound dressing adapted to facilitate re-entry to the wound. Their device includes a porous foam layer enclosed within a sheet of fenestrated elastomeric material that may be placed in direct contact with the viscera. Multiple layers of foam may be used. An adhesive elastomeric sheet is applied to cover the wound dressing and to seal in contact with the skin surrounding the incision. Suction is applied through the top sheet to drain fluids from the body cavity.

In “Vacuum pack technique of temporary abdominal closure: a 7-year experience with 112 patients,” Vol. 48, No. 2, published 2000, Journal of Trauma, Injury, Infection, and Critical Care, Barker et al. disclose a temporary abdominal closure assembly. Their assembly includes a polyethylene sheet that is perforated by the surgeon prior to being disposed between the viscera and abdominal wall. A moistened surgical towel is placed on top of that sheet and arranged to fill the incision opening. A pair of flat silicone drains is then placed on top of the towel. A covering formed from polyester sheet is adhered to the skin surrounding the incision, and vacuum is applied to the body cavity under the covering.

A V.A.C. Abdominal Dressing System is disclosed in an advertisement brochure, (c) 2003, from KCI, having a place of business at 8023 Vantage Drive, San Antonio, Tex. 78230. The V.A.C. device includes a fenestrated non-adherent layer to be placed under the fascia and over the omentum or organs. The non-adherent layer includes an encapsulated foam element adapted to minimize dressing shift and promote dressing centering. A layer of perforated foam, which may be sized in agreement with the incision opening, is installed on top of the non-adherent layer. A semiocclusive drape is applied over the perforated foam, and a vacuum is applied through a hole cut in the drape.

### DISCLOSURE OF THE INVENTION

Embodiments structured according to certain aspects of the instant disclosure provide an apparatus forming a temporary closure for a surgical access opening to a body cavity of a medical patient. The temporary closure assembly includes a base and a cap. Certain temporary closures also include a suction conduit associated with the base and capable of extracting fluids from inside the body cavity. An exemplary base carries a suction manifold disposed in fluid communication with a suction conduit for connection to a suction source.

An exemplary base includes a first transverse member adapted for substantially parallel disposition over an exterior surface of the patient effective to form an air-resistant seal with a cooperating patient surface area disposed around a perimeter of the surgical access opening. The first transverse member of the base typically includes a compliant and drapable sheet that is configured to facilitate forming an air-resistant seal around a perimeter of the access opening. A transverse member may be formed by a portion sectioned from a single sheet of drapable material. An operable sheet may also be formed by a combination of elements, such as left and right drape elements adapted to form a sealing interface with structure associated with the base. Certain bases may include a second transverse member affixed to, and extending from, structure associated with the base. In the latter case, the second transverse member is generally disposable inside the patient effective to resist adhesions between viscera and a wall of the body cavity. Sometimes, a base includes an inflatable member.

The base also typically includes a portal wall adapted for disposition in general agreement with a perimeter of the access opening. Generally, the portal wall is configured to project at an angle with respect to the first transverse member. In certain embodiments, the cap may be anchored to the portal wall. Certain portal walls may include an internally projecting sleeve having an exterior surface configured and arranged to define, at least in part, an opened or retracted shape of the access opening. Certain sleeves may include a portion that is adapted for its disposition in substantially parallel agreement with a perimeter body-edge caused by the surgical incision through a wall of the body cavity. Sometimes, a portal wall defines a perimeter of a chamber providing an additional volume into which viscera may distend. In certain cases, such chamber may include a portion disposed external to the patient's natural body cavity.

One exemplary temporary closure includes a portal wall having a portion configured as an internally projecting sleeve, with a portion of a suction manifold being carried by that sleeve. Certain suction manifolds are configured to extend substantially completely around a perimeter of the surgical opening while leaving an interior portion of the opening unobstructed to permit observation of viscera of the patient. One operable suction manifold is spaced apart from contact with viscera by a fluid permeable spacer adapted to resist damage to viscera that might result from contact between viscera and structure of the manifold.

An exemplary cap includes structure operable to substantially span the access opening effective to resist transit of ambient air therethrough. In general, a perimeter edge of the cap is anchored to structure associated with the base. Structure associated with the cap may be configured to repeatably open and close the access opening effective to permit surgical access to the cavity when the cap is open and to resist access of air into the cavity when the cap is closed. Sometimes, the cap is removable from attachment to structure associated with the base. In certain embodiments, the perimeter edge of the cap is anchored to the base, and an interior portion of the cap carries resealable structure defining an opening through which to permit repeated and resealable access to the body cavity. Certain caps include a transparent portion adapted to permit observation of viscera structure disposed thereunder. Desirably, a cap is configured and arranged to accommodate a change in size of an effective body cavity as viscera distend and relapse. A portion of certain caps may be rolled-up, effective to reduce a volume contained within those caps. Also, portions of some caps are structured to wrinkle under application of vacuum effective to reduce a volume contained within such caps.

Attachment of a cap to a portal wall may be effected by a clamping device configured to trap a perimeter edge portion of the cap in engagement against an externally projecting portion of the portal wall. Alternatively, attachment of a cap to a portal wall may be effected by a structural interference formed between structure carried at a perimeter edge of the cap and structure carried by the portal wall. Structure carried around a perimeter edge of a cap can sometimes be permanently affixed to structure associated with a base prior to installation of a base into a patient. Certain embodiments of temporary closures include a cap that is replaceable with a different cap while the base remains installed in the patient.

### BRIEF DESCRIPTION OF DRAWINGS

FIG. 1 is a view in perspective of a temporary closure system structured according to certain aspects of the instant invention that is installed in a subject (e.g., a patient).

FIG. 2 is a cross-section of a temporary closure system taken through a transverse plane similar to a plane through section T-T in FIG. 1.

FIG. 3 is a cross-section of a temporary closure system taken through a transverse plane similar to a plane through section T-T in FIG. 1.

FIG. 4 is a transverse cross-section of a workable base element.

FIG. 5 is a cross-section of a temporary closure system taken through a transverse plane similar to a plane through section T-T in FIG. 1.

FIG. 6 is a cross-section of a temporary closure system taken through a longitudinal plane similar to a plane through section L-L in FIG. 1:

FIG. 7 is a top view of a temporary closure system.

FIG. 8 is an end view in elevation of the embodiment illustrated in FIG. 7.

FIG. 9 is a side view in elevation of the embodiment illustrated in FIG. 7.

FIG. 10 is a mid-line cross-section view of the area indicated at 10 in FIG. 9.

### BEST MODE(S) FOR CARRYING OUT THE INVENTION

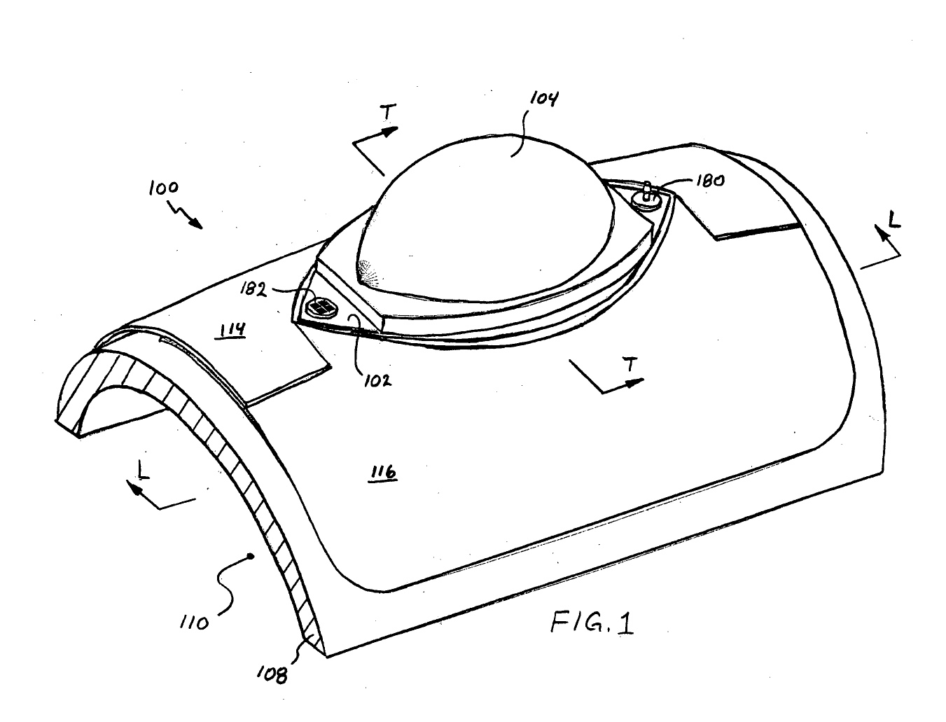

A first embodiment of a temporary closure assembly for a body cavity, which is structured according to certain principles of the instant invention, is illustrated in FIG. 1, and is indicated generally at 100. The closure assembly 100 may function as a temporary closure for a body cavity in general, including for temporarily closing a surgical entrance or opening made into an abdominal cavity, thoracic cavity, etc. For purpose of this disclosure, reference will typically be made to use of a temporary cavity closure in connection with an abdominal cavity. However, it will be understood that closure systems structured according to certain principles of the instant invention are not to be so limited.

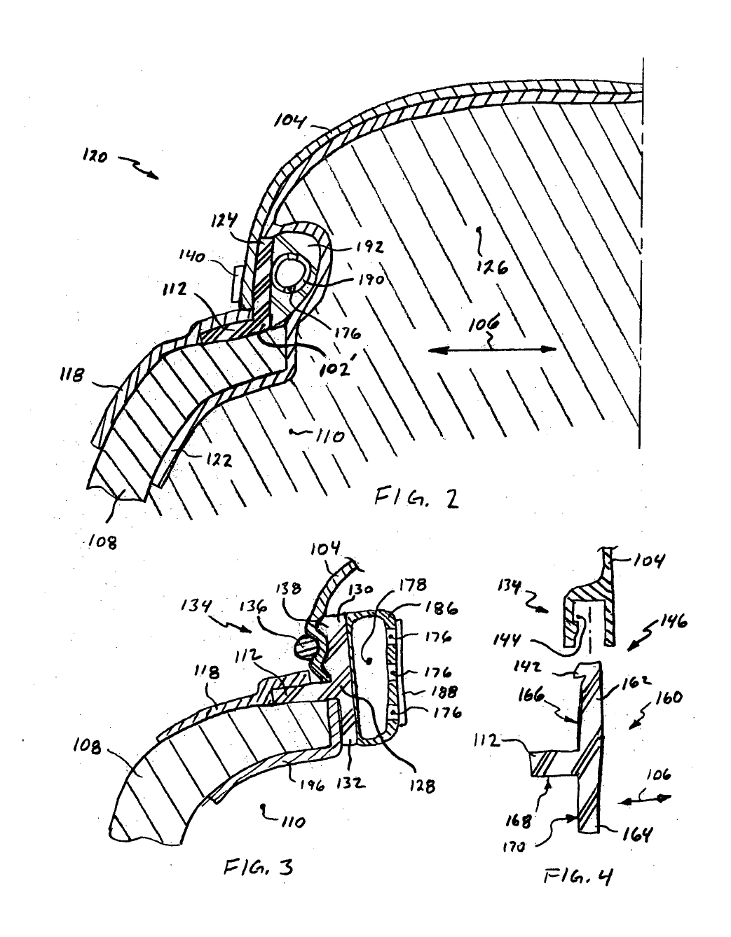

Temporary closure assembly 100 illustrated in FIG. 1 includes a base 102 and a cap 104. The cap 104 typically spans an access opening, indicated by two-headed arrow 106 in FIG. 2, through the patient's body wall, such as abdominal wall 108. Structure associated with a cap 104 desirably provides barrier structure effective to close and open the access opening 106 a plurality of times to permit repeated surgical access to the body cavity 110. When the cap 104 is closed, it is typically effective to resist transit of ambient air through the access opening 106 and into the cavity 108. Typically, as will be discussed in more detail below, a perimeter edge of the cap 104 is anchored in some way to structure associated with the base 102. Desirably, a cap 104 is structured to resist undesired loss of fluid from the cavity, and otherwise to form a protective layer for the contents of the cavity.

A base 102 desirably is structured to have a plan-form that is generally in agreement with a retracted shape of the access opening 106. In certain embodiments, the base 102 can assist in forming a retracted shape for the opening 106, or even imparting a shape to the opening. As illustrated in combination between FIGS. 1 and 2, one shape for an access opening 106 associated with a base 102, or 102′, may be characterized as being somewhat like a football in cross-section.

With reference to FIG. 2, base 102′ includes a transverse member 112 adapted for substantially parallel disposition over an exterior surface of the patient effective to form an air-resistant seal with a cooperating patient surface area (e.g., skin) disposed around a perimeter of the surgical access opening 106. Desirably, the transverse member 112 extends circumferentially around an entire perimeter of the base 102, 102′, to assist in forming an air-resistant connection disposed around a perimeter of the surgical opening.

Sometimes, the transverse member 112 may include, or be augmented by, a compliant and drapable sheet element that is configured to facilitate forming the desired air-resistant seal. Such a sheet element may be formed as a single-piece, or may include a plurality of components, such as illustrated left drape 114 and right drape 116 (see, FIG. 1). It should be realized that an operable transverse member may constitute an element that is pre-manufactured as a constituent component of the base 102, 102′. For example, a drape 118 (see, FIG. 2), which may encompass both of left drape 114 and right drape 116, may be formed as an extension to, or direct replacement of, illustrated transverse member 112. Similarly, it may be realized that an equivalent transverse member 112 may be formed during installation of the temporary closure assembly 100 in association with a patient. One such latter arrangement may be formed by bonding a drapable sheet to structure associated with a base, such as base 102, that is installed in a patient Of course, a drapable sheet element 118 may be suitably anchored to other bases having other alternative cross-section configurations, which are not illustrated, effective to form an operable transverse member.

With continued reference now to FIG. 2, it is often desirable to include a peel-ply 122 as a spacer element disposed over the opening 106 to resist imparting contact-induced damage from a temporary closure assembly to the viscera. The peel-ply 122 may also be arranged to extend transversely and longitudinally to form a barrier between the wall 108 and viscera, as illustrated. Such a peel-ply 122 desirably is effective to resist formation of adhesions between the viscera and either the abdominal wall or closure structure. Certain peel-plies 122 also include portions that are substantially transparent, to permit visual observation of the viscera when a closure, such as closure assembly 120, is installed. Furthermore, the peel-ply 122, if present in a temporary closure assembly, desirably is perforated, or otherwise permeable to fluids, to assist in extraction of excess fluid from the cavity 110.

The base 102′ illustrated in FIG. 2 includes a portal wall 124 that is configured to project at an angle with respect to transverse member 112. As illustrated, the angle between portal wall 124 and transverse member 112 is approximately orthogonal, but a range of other angles are workable, and the angle may vary around a perimeter of the base. Portal wall 124 extends around the perimeter of an opening passing through base 102′, and is generally sized and configured for its disposition in proximity to the perimeter of an access opening 106. The portal wall 124 may be characterized as forming an externally projecting sleeve through which access may be obtained into the cavity 110. As illustrated in FIG. 2, portal wall 124 defines a perimeter of a chamber 126 providing an additional volume into which viscera may distend. Chamber 126 illustrated in FIG. 2 comprises a portion of volume disposed external to the patient's natural body cavity 110.

With reference now to FIG. 3, base 128 includes a transverse member 112 disposed at an angle to, and intersecting with, externally projecting sleeve 130 and internally projecting sleeve 132. Collectively, externally projecting sleeve 130 and internally projecting sleeve 132 form a portal wall member of base 128. A proximal perimeter edge portion, generally indicated at 134, of a cap 104 may be anchored to structure associated with base 128. As illustrated in FIG. 3, edge 134 is anchored to the portal wall portion of base 128. The anchoring attachment illustrated in FIG. 3 is removable to permit replacement of cap 104 with a different cap 104 having the same or different size or configuration, or simply to provide repeatably sealable surgical access through opening 106 to the cavity 110. In certain embodiments, a cap 104 may be replaced with a different cap while a base 102, 128 remains installed in a patient.

The anchoring attachment for cap 104 illustrated in FIG. 3 is formed by trapping edge 134 between a clamping device, such as elastomeric O-ring 136, and cooperating structure 138 of the externally projecting sleeve 130. Presence of cooperating structure, such as structure 138 that is configured in harmony with the O-ring 136, may be in a clamp-anchor type arrangement to resist undesired decoupling of the air-resistant seal formed at the joint between cap 104 and a base, such as base 128. However, for simplicity of certain illustrations, and where joint reinforcement structure is not required, illustration of such structure is sometimes omitted (e.g., FIG. 2).

An alternative workable clamping arrangement, illustrated in FIG. 2, includes band 140 that may be formed from an elastomeric element, like a rubber band. In another workable arrangement to anchor a cap 104, band 140 may be a less-elastic element, such as a steel band portion of a clamping element, which may be reduced in effective size/diameter to trap a wall portion of cap 104 against structure associated with base 102′ by operating an over-center toggle-clamp mechanism of any well known type (not illustrated). Other clamping devices are also operable to secure a cap in association with a base, nonexclusively including plastic tie elements, such as those commonly associated with electric wiring harnesses and generally permitting a one-way size adjustment.

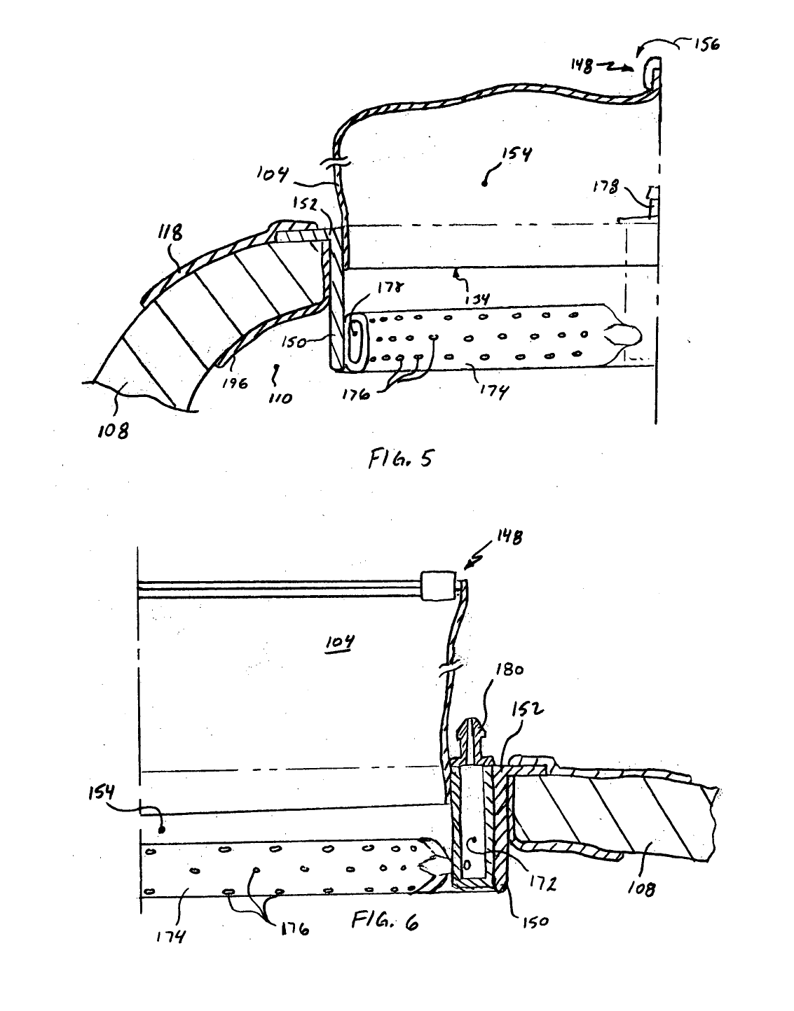

Among other workable alternatives, a cap 104 may be affixed to a base using any other known operable manufacturing technique and structural arrangement, nonexclusively including: adhesive; chemical, acoustic, and/or thermal welding; and structural interference fit, such as may be formed between tooth 142 and socket 144 of the snap-together anchor structure generally indicated at 146 in FIG. 4. In general, the interface between cap and base simply provides an air-resistant connection to resist transit of ambient air into the body cavity 110. A perimeter edge of a cap may even be affixed to structure associated with a base by application of adhesive tape installed as an alternative clamping element, or as a bridge element configured to overlap a portion of each of cap edge 134 and cooperating structure associated with a base. It should be noted that, in general, caps may either be permanently affixed, or be removably affixed, to structure associated with a base, such as base 102, 128. Sometimes, a cap may be permanently affixed to a base during manufacture of a temporary closure assembly.

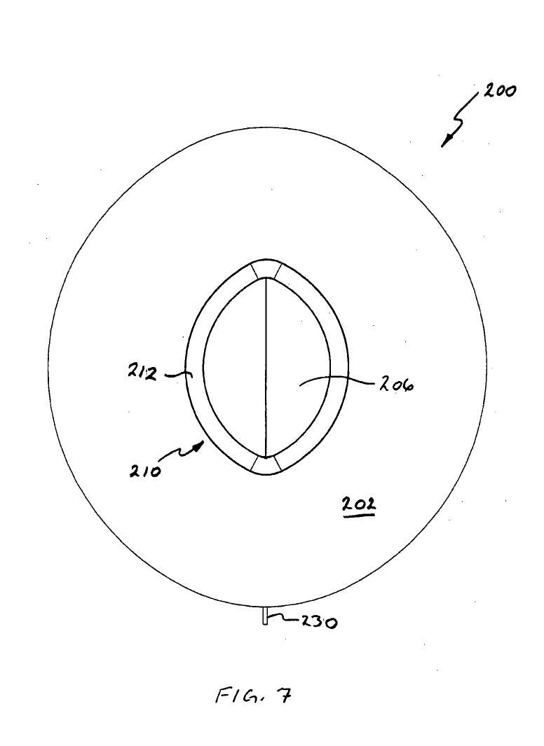

Cap 104 illustrated in FIGS. 5 and 6 includes an interior portion carrying a resealable structure, generally indicated at 148, defining a resealable opening through which to permit repeated and resealable access to cavity 110. The illustrated resealable structure 148 is commonly called a zipper closure, such as is contained in certain bags made from a plastic film and commercially available under the ZIPLOCK™ brand. A bottom may be removed from such a plastic bag, and the resulting newly formed edge 134 may be affixed to a base, to make an exemplary workable cap 104. It is within contemplation that a cap 104 can be resealable around a perimeter edge 134, as well as providing interior resealable structure 148. As further illustrated in FIGS. 5 and 6, a perimeter edge 134 of cap 104 may be permanently anchored to the portal wall 150.

Certain embodiments of a cap 104 desirably include a transparent portion adapted to permit observation of structure disposed thereunder. In general, it is also desirable that a cap 104 be configured and arranged to accommodate a change in effective size of a cavity 154 that is partially bounded by the cap as viscera distend and relapse. A portion of certain caps 104 may be rolled-up (e.g., starting at closure 148, see arrow 156 in FIG. 5), effective to reduce a volume contained within the cap. A portion of certain caps 104 may also be structured to wrinkle under application of vacuum effective to reduce a volume contained within the cap.

Caps 104 that are transversely flexible may desirably provide a traveling boundary that can displace responsive to changes in size of the volume occupied by viscera. In such case, as the viscera shed excess fluid and return to their homeostatic condition, a cap 104 included in certain embodiments may visually present a concavity (in contrast to the convex shape presented in FIGS. 1 and 2) indicating status of such return. Such a status indicator can be helpful in evaluating progress toward the patient state required to permanently reclose the surgical opening.

With reference again to FIG. 4, the portal wall, generally indicated at 160, includes an externally projecting sleeve 162 and an internally projecting sleeve 164. Each of such sleeves projects at an angle with respect to transverse member 112. Sleeve 162 and 164 are illustrated in FIG. 4 as being substantially in-line, although such is not required. Further, each sleeve 162, 164 may be oriented at any individually workable angle with respect to transverse member 112, and such angles may vary around the perimeter of the base. As illustrated in FIG. 2 and FIG. 5, the presence of either of sleeve 162 or 164 is optional. It is within contemplation that a workable closure may not even include a projecting sleeve at all. In such case, portal wall structure associated with transverse member 112 may be employed to anchor a cap 104.

Still with reference to FIG. 4, external surface 166 of externally projecting sleeve 162 provides structure against which a perimeter edge of a cap may be anchored (e.g., see FIGS. 2 and 3). Similarly, in an alternative arrangement within contemplation, the opposite, internal surface of sleeve 162 may be employed as a surface against which to clampingly trap perimeter edge 134 by an internally disposed, and self-biased, cork-like stopper. One workable such cork-like stopper may be arranged as a ring-like element having an open interior through which the viscera may be observed. Also, an edge 134 may be permanently affixed to either external surface 166, or its internal counterpart, similar to the arrangement illustrated in FIG. 5.

Surface 168 of drapable transverse member 112 illustrated in FIG. 5 may be placed into sealing contact with a patient's skin over an area disposed around the surgical opening to the body cavity effective to form an air-resistant seal around a wound boundary. Surface 170 of internally projecting sleeve 164 is typically adapted for disposition in substantially parallel agreement with a perimeter body-edge caused by the wound incision through a wall of the patient's body cavity. It is within contemplation that exterior surface 170 may be configured and arranged to define, at least in part, a shape of the access opening 106.

With reference now to FIG. 6, a suction conduit 172 in association with a base, such as base 152 may be included. The illustrated suction conduit 172 is configured and arranged to assist in extracting fluids from inside the cavity 110. As further illustrated, base 152 carries a suction manifold 174 disposed in fluid communication with conduit 172. One operable manifold 174 may be formed by adapting a conventional surgical drain, such as a substantially flat silicone drain, to be carried on a base 152 and for the plurality of relatively small-in-size drain openings 176 to be placed into communication through intermediate lumen 178 to conduit 172 for fluid evacuation from the cavity 110. Connection structure, such as nipple 180, may be provided as a convenient attach point for communication from conduit 172 to a suction source.

As illustrated in FIG. 1, a suction attach structure 180 may be disposed in proximity to an edge of a base 102. Furthermore, a plurality of such attach structures may be included in certain embodiments of temporary closures. The sealing cap 182 is removable and replaceable with an attach structure 180 effective to provide an alternative fluid communication path to cavity 110. Therefore, suction may be applied to either end, or simultaneously to both ends, of the surgical incision, as may desired in a particular care-giving situation.

With reference to FIGS. 5 and 6, portal wall 150 provides an internally projecting sleeve on which is carried a portion of suction manifold 174. In certain cases, such as where the patient has a thick body wall, it can be helpful to dispose the suction manifold 174 at a relatively deeper position in cavity 110 (e.g., as compared to the external and shallow arrangements illustrated in FIGS. 2 and 3, respectively). Illustrated suction manifold 174 is configured to extend substantially completely around a perimeter of the surgical opening, while desirably leaving an interior portion of that opening unobstructed to permit observation of viscera of the patient.

All surfaces of a temporary closure may be configured to resist imparting contact-induced damage to the patient, and particularly to the patient's viscera. For example, relatively large-in-size drain apertures 176 of suction manifold 186 illustrated in FIG. 3 are spaced apart from contact with viscera by a fluid permeable spacer 188 that is adapted to resist damage to viscera while permitting extraction of fluid from cavity 110. Drain apertures 176 of suction manifold 190 illustrated in FIG. 2 are spaced apart from contact with viscera by a fluid permeable spacer 192 that is adapted to resist damage to viscera while permitting extraction of fluid from cavity 110. Spacer 192 may be formed from open-celled foam, or other suitable fluid-permeable material. The illustrated shape of spacer 192 is also effective to provide a desired large surface area over which unobstructed suction may be applied to the viscera to assist in evacuating fluid from the cavity 110.

As illustrated in FIGS. 3 and 5, a second transverse member 196 may be carried in association with a base 128, 152. The anchoring attachment between transverse member 196 and its associated base desirably provides an air-resistant seal between such components. Furthermore, the configuration of the second transverse member 196 desirably provides a somewhat automatic accommodation for walls 108 having different thicknesses. As illustrated, member 196 may flare away from the portal wall in agreement with an inside surface of the body wall 108. If the wall 108 of a patient is thicker, an additional length of member 196 simply fits against the portal wall like a turtle-neck shirt extension. As shown in FIG. 3, the extension of member 196 may even extend internally beyond the termination of the portal wall sleeve 132, if required (e.g., to accommodate an obese patient).

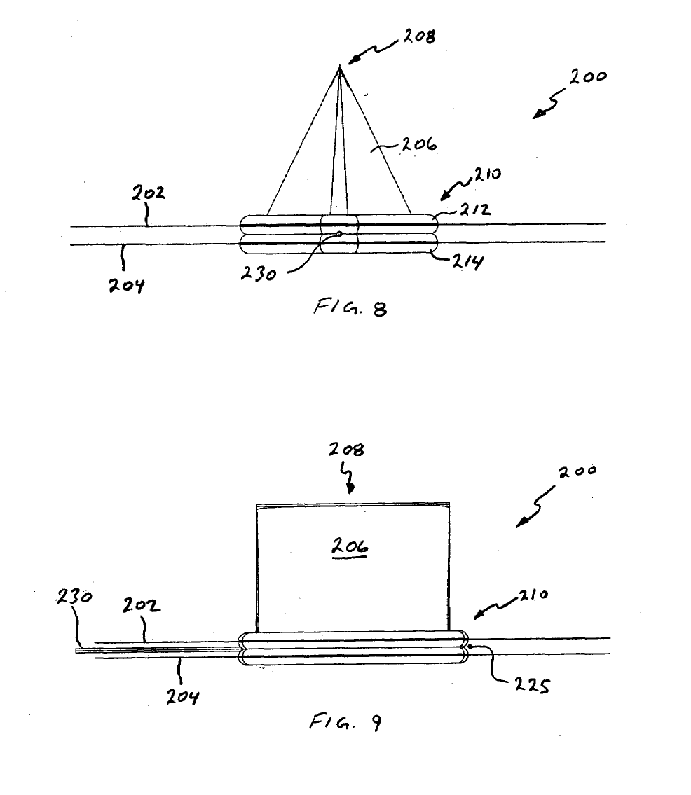

Another temporary closure embodiment is indicated generally at 200 in FIGS. 7-10. Closure 200 includes a top drape 202 and a bottom drape 204, which function similarly to previously described drapes. A collapsible envelope 206 provides a reclosable cap for the surgical opening, and forms a conveniently adjustable-sized extension to the patient's body cavity into which viscera may distend. The transversely flexible envelope 206 provides a moving boundary that travels with the viscera as excess fluid is removed and swelling is reduced. Desirably, the envelope 206 is sufficiently transparent as to permit observation of the viscera when the envelope 206 is closed. The reclosable opening, generally indicated at 208, may be maintained in a closed configuration by way of a ZIPLOCK™ type interlocking seam, rolled and tied like a dry-bag, or may be sealed in any other operable manner that will be apparent to one of ordinary skill in the art.

Base 210 includes a top inflatable tube 212, and a bottom inflatable tube 214. Tubes 212 and 214 are desirably at least somewhat transversely compliant to accommodate to changes in curvature between bodies of different patients. The closure 200 is typically shipped and stored in a deflated state. Inflation valve 220 permits inflation of base 210 with a gas, typically air, during installation of the closure 200 in a patient. In the illustrated embodiment, a single inflation valve 220 permits communication of gas into top tube 212. Tube 212 communicates the gas to tube 214 to effect inflation of both tubes. However, it is within contemplation to provide separate inflation structure for each tube. In such case, the pressure in each tube may be separately controlled.

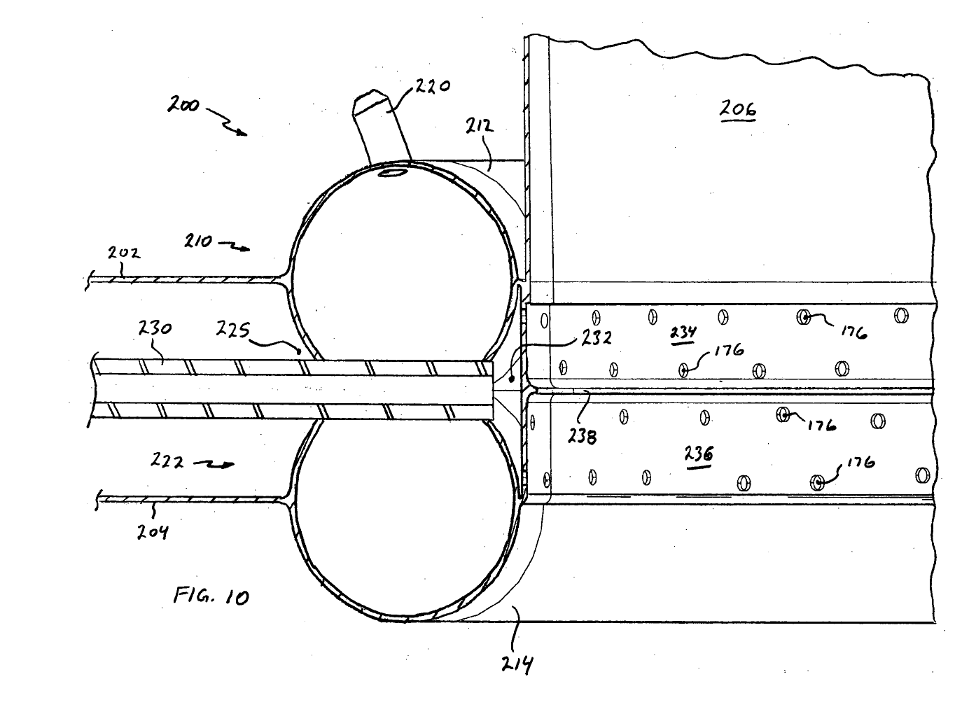

During installation, the bottom drape 204 is tucked inside the body cavity, and smoothed against the interior body wall inside and around the perimeter of the surgical opening. Inflation of the tubes 212, 214, tends to bias the portal wall, generally 222 (see, FIG. 10), of base 210 into registration with the body wall around the perimeter of the surgical opening. The notch 225 (see, FIGS. 9 and 10) between top tube 212 and bottom tube 214 forms receiving structure into which the body wall is engaged around the perimeter of the surgical opening. The notch 225 is therefore effective to assist in maintaining the closure 200 in an installed position, and to resist undesired “popping out” of the closure. A size of the base may be adjusted to fit a given perimeter size, to a certain extent, by increasing or decreasing the pressure in the tubes 212, 214. The pressure may also be manipulated to accommodate the base 210 to body walls of different thickness. The top drape 202 is typically bonded to the skin of the patient using a surgical-grade adhesive.

Suction line 230 is disposed to extract fluid from drain sump 232 (see, FIG. 10). As illustrated, suction line 230 is routed between both drapes 202, 204, although it is contemplated that it will be routed through the top drape near the top tube 212, or even through the tube 212 directly to bypass the top drape 202 completely. The contemplated routing facilitates making an air-resistant seal between top drape 202 and the skin of a patient. It is also within contemplation to provide one or more additional suction line to extract fluid from the drain sump 232.

The top drape 202 and tube 212 of the illustrated closure 200 are made by bonding a pair of medical-grade urethane sheets together to form a two-ply drape 202 and single-ply inflatable tube 212. The bottom drape 204 and tube 214 are similarly made. Base 210 is constructed by bonding the top tube 202 to the bottom tube 204 around their adjacent perimeter to form a wall for drain sump 232. Drain apertures 176 are die-cut, or otherwise formed, in the two-ply material 234, 236 inside the respective top and bottom tubes. The top material 234 is then bonded to the bottom material 236 at seam 238 to form the opposite wall of drain sump 232. Envelope 206 may be bonded, or otherwise affixed, to a portion of top material 234, as illustrated, or to other convenient structure. It is currently preferred to bond certain structure of the closure 200 using radio frequency welding.

A temporary closure assembly structured according to certain principles of the instant invention will typically be made from medical grade plastic, and plastic-like materials, such as urethane, polyurethane, TEFLON™, and the like. Selection, structuring, arrangement, and integration of the appropriate constituent materials may be made in accordance with conventional manufacturing principles and procedures commonly employed in the manufacture of medical devices.

1. An apparatus adapted to provide a temporary closure for a surgical incision that provides an access opening into a body cavity in a medical patient, the apparatus comprising:
a base comprising:
a first transverse member adapted for substantially parallel disposition over an exterior surface of the patient effective to form an air-resistant seal with a cooperating patient surface area disposed around a perimeter of the access opening; and
a portal wall adapted for disposition in general agreement with a perimeter of the access opening; and
a cap operable to substantially span the access opening effective to resist transit of ambient air therethrough, with a perimeter edge of the cap being anchored to structure associated with the base.

2. The apparatus of claim 1, further comprising:
a suction conduit associated with the base and capable of extracting fluids from inside the cavity.

3. The apparatus of claim 1, wherein:
structure associated with the cap is configured to repeatably open and close the access opening effective to permit surgical access to the cavity when the cap is open and to resist access of air to the cavity when the cap is closed.

4. The apparatus of claim 3, wherein:
the cap is removable from attachment to structure associated with the base.

5. The apparatus of claim 3, wherein:
a perimeter edge of the cap is anchored to the base; and
an interior portion of the cap carries resealable structure defining an opening through which to permit repeated and resealable access to the cavity.

6. The apparatus of claim 5, wherein:
the portal wall is configured to project at an angle with respect to the first transverse member; and
the cap is anchored to the portal wall.

7. The apparatus of claim 3, wherein:
the cap comprises a transparent portion adapted to permit observation of structure disposed thereunder.

8. The apparatus of claim 3, wherein:
the cap is configured and arranged to accommodate a change in effective size of the cavity as viscera distend and relapse.

9. The apparatus of claim 8, wherein:
a portion of the cap may be rolled-up, effective to reduce a volume contained within the cap.

10. The apparatus of claim 8, wherein:
a portion of the cap is structured to wrinkle under application of vacuum effective to reduce a volume contained within the cap.

11. The apparatus of claim 4, wherein:
attachment of the cap to the portal wall is effected by a clamping device configured to trap a perimeter edge portion of the cap in engagement against an externally projecting portion of the portal wall.

12. The apparatus of claim 4, wherein:
attachment of the cap to the portal wall is effected by a structural interference formed between structure carried at a perimeter edge of the cap and structure carried by the portal wall.

13. The apparatus of claim 4, wherein:
the cap is replaceable with a different cap while the base remains installed in the patient.

14. The apparatus of claim 3, wherein:
structure carried at a proximal perimeter edge of the cap is permanently affixed to structure associated with the base prior to installation of the base into the patient.

15. The apparatus of claim 1, wherein:
the portal wall comprises an internally projecting sleeve having an exterior surface configured and arranged to define, at least in part, a retracted shape of the access opening.

16. The apparatus of claim 15, wherein:
the portal wall defines a perimeter of a chamber providing an additional volume into which viscera may distend.

17. The apparatus of claim 16, wherein:
the chamber comprises a portion disposed external to the patient's natural body cavity.

18. The apparatus of claim 15, wherein:
the sleeve is adapted for disposition in substantially parallel agreement with a perimeter body-edge caused by the incision through a wall of the body cavity.

19. The apparatus of claim 2, wherein:
the base carries a suction manifold disposed in fluid communication with the conduit.

20. The apparatus of claim 19, wherein:
the portal wall comprises an internally projecting sleeve; and
a portion of the suction manifold is carried by the sleeve.

21. The apparatus of claim 19, wherein:
an interior portion of the cap carries resealable structure defining an opening through which to permit repeated and resealable access to the cavity; and
the suction manifold is configured to extend substantially completely around a perimeter of the opening while leaving an interior portion of the opening unobstructed to permit observation of viscera of the patient.

22. The apparatus of claim 19, wherein:
the suction manifold is spaced apart from contact with the viscera by a fluid permeable spacer adapted to resist damage to the viscera that might result from contact between the viscera and structure of the manifold.

23. The apparatus of claim 1, further comprising:
a second transverse member affixed to, and extending from, structure associated with the base, the second transverse member being disposable inside the patient effective to resist adhesions between viscera and a wall of the body cavity.

24. The apparatus of claim 1, wherein:
the first transverse member of the base comprises a compliant and drapable sheet configured to facilitate forming the air-resistant seal.

25. The apparatus of claim 24, wherein:
the sheet comprises left and right drape elements adapted to form a sealing interface with structure associated with the base.

26. An apparatus adapted to provide a temporary closure for a surgical incision that provides an access opening into a body cavity in a medical patient, the apparatus comprising:
a base comprising:
a first transverse member adapted for substantially parallel disposition over an exterior surface of the patient effective to form an air-resistant seal with a cooperating patient surface area disposed around a perimeter of the access opening; and
a portal wall adapted for disposition in general agreement with a perimeter of the access opening, the portal wall comprising a sleeve having an exterior surface configured and arranged to define, at least in part, a retracted shape of the access opening, the sleeve projecting at an angle with respect to the first transverse member and being oriented to extend internally with respect to the patient; and
a cap configured to substantially span the access opening and adapted for anchoring a perimeter edge of the cap to structure associated with the base, the cap being configured to repeatably close and open the access opening effective to permit surgical access to the cavity when the cap is open and to resist access of ambient air into the cavity when the cap is closed.

27. The apparatus of claim 26, further comprising:
a suction manifold carried by the sleeve and adapted to remove fluid from the cavity, the suction manifold being configured to extend substantially completely around a perimeter of the access opening while leaving an interior portion of the opening unobstructed to permit observation of viscera of the patient.

28. The apparatus of claim 26, wherein:
the base comprises an inflatable member.

29. The apparatus of claim 1, wherein the base is inflatable.
